(19) United States
(12) Patent Application Publication
(10) Pub. No.: US 2005/0279368 A1
(43) Pub. Date: Dec. 22, 2005

(54) Computer assisted surgery input/output systems and processes

(76) Inventor: Daniel L. McCombs, Germantown, TN (US)

Correspondence Address: CHIEF PATENT COUNSEL;SMITH & NEPHEW, INC., 1450 BROOKS ROAD, MEMPHIS, TN, 38116, US

(21) Appl. No.: 10/869,785

(22) Filed: Jun. 16, 2004

(51) Int. Cl. A61B 19/00; A61B 17/00; H04N 7/18; H04N 9/47

(52) U.S. Cl. 128/897; 606/1; 348/77

(57) ABSTRACT

Input/Output systems and processes for computer assisted surgery systems are described. In one embodiment, a rendering apparatus is adapted to render display information on a presentation substrate during surgery. A plurality of location indicia are attached to the presentation substrate and a sensor senses the position and orientation of the location indicia. A computer functionality determines the position and orientation of the presentation substrate from the information from the sensor on the position of the location indicia. The computer functionality coordinates the rendering apparatus with the position of the display substrate so that the rendering apparatus renders display information onto the presentation substrate used in surgery. In another embodiment, the rendering apparatus renders interaction indicia onto a presentation substrate and a monitoring apparatus monitors interaction with the interaction indicia to allow data to be input into the computer functionality by way of the interaction indicia.

### FIELD OF THE INVENTION

The present invention relates to computer assisted surgical systems. More specifically, the invention relates to rendering display information such as images generated by such systems, and, in certain cases, interaction indicia, such as menus or control buttons for entry of commands or other information into such systems, on presentation substrates located at or near the surgical site.

### BACKGROUND

Computer assisted surgery offers significant advantages over conventional surgery, because it enables the generation and display of real time images which show, among other things, internal anatomical structures in spatial relationship with items which are in use during the surgery. These items may include surgical instruments, surgical implants, and parts of the body on which surgery is being conducted. Such systems also typically generate and display textual information such as orientation information, instructions, and other information which is useful in the surgical process. One disadvantage in conventional computer assisted surgery, however, is that in order to view the information displayed by a conventional computer assisted surgery monitor, the surgeon must divert her gaze from the site of the surgery and lose continuity of the surgical process. This loss frequently entails the surgeon shifting attention and focus away from the surgical site and the consequent need to reestablish bearings when directing attention back to the surgical site. Having to shift focus from the surgical site to a monitor and back is inconvenient for the surgeon, and among other things increases the time required for the surgical procedure, and increases the likelihood of surgical error.

Attempts have been made to display information on an eye piece worn by the surgeon or on a semi-transparent screen between the surgeon and the patient. These methods, however, are cumbersome and partially obstruct the surgeon's view. Moreover, they introduce additional items into the surgical procedure which increases the instrument count and increases the danger of contamination. Other efforts include voice recognition technology, which involves latency issues, the need to confirm commands, and potential inaccuracies and errors that can occur because of the conventional shortcomings which continue to impair use of speech recognition technology in general.

An additional problem with conventional computer assisted surgery input and output functionality is that in order to enter data into the computer system, a surgeon must use a data input device such as a keyboard or mouse, sometimes in combination with a pedal. These data input devices further increase the risk of contamination and make entering data cumbersome, distracting, time consuming and open to potential errors.

Therefore, the need exists for displaying and entering data from and to computer assisted surgery systems in a manner that, among other things, avoids requiring the surgeon to divert attention or focus from the surgical site, reduces the possibility of contamination, and increases speed, accuracy and reliability of data output and input to the computer assisted surgery systems.

### SUMMARY

Systems and processes according to certain embodiments of the present invention allow a surgeon to receive display information from the computer assisted surgery system and to enter commands and other information into the computer assisted surgery system using presentation substrates that may be located at or near the surgery site. Such substrates can include (i) a body part, (ii) a surgical device such as an instrument, an implant, a trial or other surgical device, and/or (iii) another substrate such as sheet or a screen positioned on the patient or operating table. Such substrates are tracked in position by the computer assisted surgery system so that the projector or other rendering apparatus for rendering the display information and monitoring the surgeon's interaction with the input indicia can track that position and orientation and allow rendering to occur as the substrate moves and changes in orientation. Systems and processes according to various embodiments of the invention accordingly eliminate the need for the surgeon to divert attention or focus from the surgical site in order to see the display information or interact with the input indicia, among other benefits and advantages.

According to certain aspects of the invention, display information may be rendered using laser display apparatus devices, optical devices, projection devices, or other desired techniques. Such display information can include conventional computer assisted surgery graphical information, text, menus, and other presentations. Input indicia such as menus, buttons, and other selection items can be displayed and interaction with them monitored by an interaction monitoring apparatus such as the rendering device or another device associated with the computer assisted surgery system to cause the computer assisted surgery system to register when the surgeon has interacted to input information or a command in the system.

In systems that display the input indicia on surgical devices, because the position of the menu items is sensed and recorded in the computer functionality and because the position of the surgical instrument or other item used in surgery is sensed by the computer functionality, the surgeon may make selections from the pull down menus, menu choices, buttons, or other items by positioning the surgical instrument to correspond to the desired choice.

According to one aspect of the invention, there is provided a computer assisted surgery system including sensor apparatus for sensing position and orientation of a plurality of location indicia to which surgical items and body parts are connected and computer functionality for tracking said position and orientation of said surgical items and body parts, the system further comprising: rendering apparatus associated with the computer functionality, the rendering apparatus adapted to render display information on a presentation substrate, the presentation substrate connected to at least one location indicium adapted to be tracked by said sensor apparatus, wherein the computer functionality uses information from the sensor apparatus to track the position and orientation of the presentation substrate and cause the rendering apparatus to display the display information on the presentation substrate as the presentation substrate moves and is sensed by the sensor apparatus.

According to further aspects of the invention, the presentation substrate may comprise a body part, surgical instrument, or a display surface. According to other aspects of the invention, the rendering apparatus may be further adapted to display a plurality of interaction indicia on the presentation substrate, wherein the computer functionality further uses information from the sensor apparatus to track the position and orientation of the presentation substrate and cause the rendering apparatus to display the interaction indicia on the presentation substrate as the presentation substrate moves and is sensed by the sensor apparatus; and further comprising: a monitoring apparatus associated with the computer assisted surgery system adapted to monitor interaction with the interaction indicia, wherein the computer functionality further causes the monitoring apparatus to track position and location of the interaction indicia in order to monitor interaction with interaction indicia.

According to other aspects of the invention, the rendering apparatus may comprise the monitoring apparatus or be separate from the monitoring apparatus. According to other aspects of the present invention, the location indicia may be fiducials. According to other aspects of the present invention, the rendering apparatus can include a laser projector and can display a graphical user interface, which can include at least one pull down menu, and/or at least one button, and/or an arrangement of letters, and/or an arrangement of numbers.

According to another aspect of the invention, there is provided a computer assisted surgery system including sensor apparatus for sensing position and orientation of a plurality of location indicia to which surgical items and body parts are connected and computer functionality for tracking said position and orientation of said surgical items and body parts, the system further comprising: rendering apparatus associated with the computer functionality, the rendering apparatus adapted to render interaction indicia on a presentation substrate during surgery, the presentation substrate connected to at least one location indicium adapted to be tracked by said sensor apparatus, wherein the computer functionality uses information from the sensor apparatus to track the position and orientation of the presentation substrate and cause the rendering apparatus to display the interaction indicia on the presentation substrate as the presentation substrate moves and is sensed by the sensor apparatus; and a monitoring apparatus associated with the computer assisted surgery system and adapted to monitor interaction with the interaction indicia, wherein the computer functionality further causes the monitoring apparatus to track position and location of the interaction indicia in order to monitor interaction with the interaction indicia.

According to another aspect of the invention, there is provided a computer assisted surgery system including a rendering apparatus associated with the computer functionality, the rendering apparatus adapted to render display information and interaction indicia on a presentation substrate; a first plurality of location indicia attached to the presentation substrate; a second plurality of location indicia attached to an item used in surgery; and a sensor apparatus adapted to sense position and orientation of the rendering apparatus, the position and orientation of the first plurality of location indicia attached to the presentation substrate; and the position and orientation of the second plurality of indicia attached to the item used in surgery, wherein the position and orientation of the rendering apparatus is coordinated with the position and orientation of the presentation substrate so that the display information and interaction indicia can be rendered on the presentation substrate, and wherein the position of the item used in surgery relative to the interaction inputs data to the computer functionality.

According to another aspect of the invention, there is provided a method comprising providing a computer assisted surgery system including sensor apparatus for sensing position and orientation of a plurality of location indicia to which surgical items and body parts are connected and computer functionality for tracking said position and orientation of said surgical items and body parts; providing a rendering apparatus associated with the computer functionality, the rendering apparatus adapted to render display information on a presentation substrate, the presentation substrate connected to at least one location indicium adapted to be tracked by said sensor apparatus, wherein the computer functionality uses information from the sensor apparatus to track the position and orientation of the presentation substrate and cause the rendering apparatus to display the display information on the presentation substrate as the presentation substrate moves and is sensed by the sensor apparatus; referencing the display information from the rendering apparatus to receive data during a surgical procedure; and completing the surgical procedure based in part on the data received from the displaying functionality.

According to another aspect of the invention, there is provided a method comprising providing a computer assisted surgery system including sensor apparatus for sensing position and orientation of a plurality of location indicia to which surgical items and body parts are connected and computer functionality for tracking said position and orientation of said surgical items and body parts; providing a rendering apparatus associated with the computer functionality, the rendering apparatus adapted to render interaction indicia on a presentation substrate during surgery, the presentation substrate connected to at least one location indicium adapted to be tracked by said sensor apparatus, wherein the computer functionality uses information from the sensor apparatus to track the position and orientation of the presentation substrate and cause the rendering apparatus to display the interaction indicia on the presentation substrate as the presentation substrate moves and is sensed by the sensor apparatus; providing a monitoring apparatus associated with the computer assisted surgery system and adapted to monitor interaction with the interaction indicia, wherein the computer functionality further causes the monitoring apparatus to track position and location of the interaction indicia in order to monitor interaction with the interaction indicia; communicating data to the computer functionality during a surgical procedure based at least on part on positioning one of the surgical items connected to a plurality of location indicia to correspond with one or more interaction indicia; and completing the surgical procedure based at least in part on the data communicated to the computer functionality.

Objects, features, and advantages of certain systems and processes according to certain embodiments of the invention include, but are not limited to one or more, or combinations of, any of the following, with or without other objects, features and advantages: reduction of need for surgeon or others to divert attention or visual focus from the surgical site; reduction of contamination possibility, and increased speed, accuracy and reliability of data output and input to computer assisted surgery systems, and control and effectiveness of such systems. Other objects, features and advantages will be apparent with respect to the remainder of this document.

### BRIEF DESCRIPTION

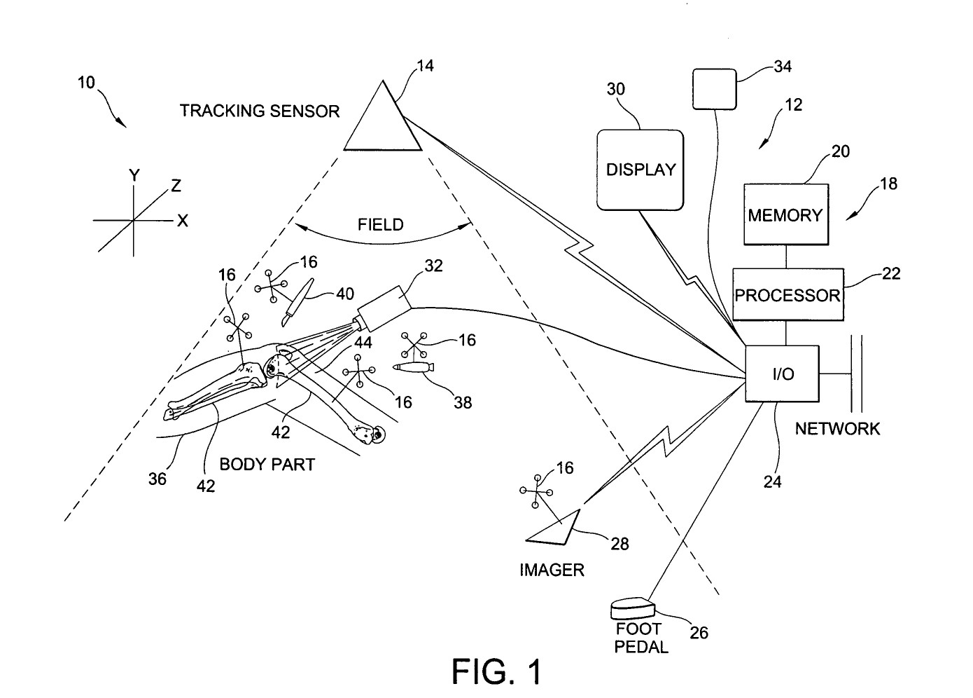

FIG. 1 is a schematic view of a computer assisted surgery system with which apparatus and processes according to aspects of the present invention may be used.

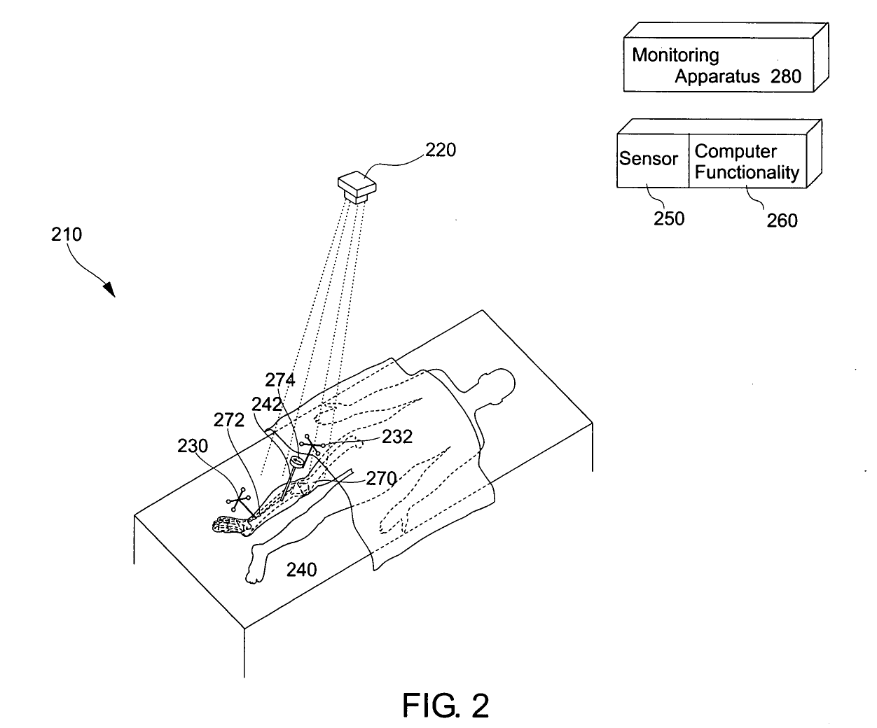

FIG. 2 is a schematic view of a computer assisted surgery system employing apparatus and processes according to one embodiment of the present invention.

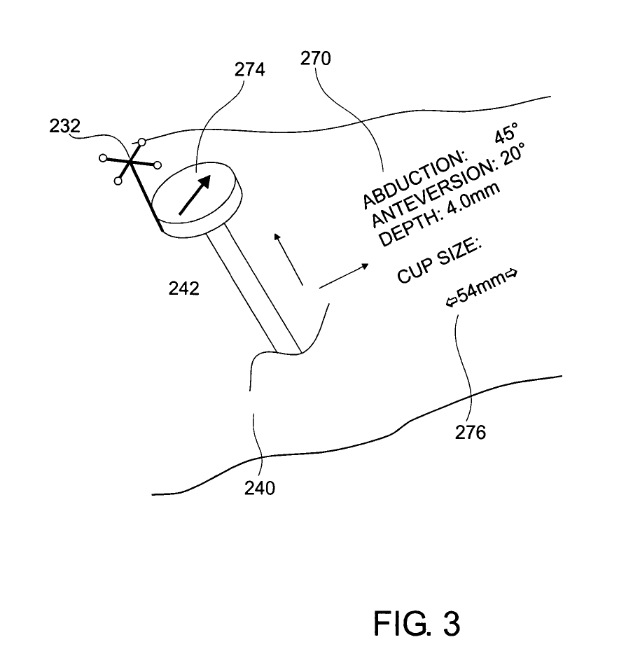

FIG. 3 is a more detailed schematic view of one aspect of the computer assisted surgery system illustrated in FIG. 2.

### DETAILED DESCRIPTION

FIGS. 2 and 3 illustrate a system according to one embodiment of the present invention. Systems according to certain embodiments of the invention as shown in FIG. 2, are adapted to be used with, as part of, or to supplement a computer assisted surgery systems which may be conventional. A conventional computer aided surgery system as used with apparatus and methods according to aspects of the invention is illustrated in FIG. 1 and may comprise a computer capacity, including standalone and/or networked, to store data regarding spatial aspects of surgically related items and virtual constructs or references including body parts, implements, instrumentation, trial components, prosthetic components and rotational axes of body parts. Any or all of these may be physically or virtually connected to or incorporate any desired form of mark, structure, component, or other location indicium or reference device or technique which allows position and/or orientation of the item to which it is attached to be sensed and tracked, preferably in three dimensions of translation and three degrees of rotation as well as in time if desired. In the preferred embodiment, such “location indicia” are reference frames each containing at least three, preferably four, sometimes more, reflective elements such as spheres reflective of lightwave, infrared, radiofrequency and/or other forms of electromagnetic energy, or active elements such as LEDs or radiofrequency devices.

Systems and processes for accomplishing computer assisted surgery are disclosed in U.S. Ser. No. 10/084,012, filed Feb. 27, 2002 and entitled “Total Knee Arthroplasty Systems and Processes”; U.S. Ser. No. 10/084,278, filed Feb. 27, 2002 and entitled “Surgical Navigation Systems and Processes for Unicompartmental Knee Arthroplasty”; U.S. Ser. No. 10/084,291, filed Feb. 27, 2002 and entitled “Surgical Navigation Systems and Processes for High Tibial Osteotomy”; International Application No. US02/05955, filed Feb. 27, 2002 and entitled “Total Knee Arthroplasty Systems and Processes”; International Application No. US02/05956, filed Feb. 27, 2002 and entitled “Surgical Navigation Systems and Processes for Unicompartmental Knee Arthroplasty”; International Application No. US02/05783 entitled “Surgical Navigation Systems and Processes for High Tibial Osteotomy”; U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; U.S. Ser. No. 10/229,372, filed Aug. 27, 2002 and entitled “Image Computer Assisted Knee Arthroplasty”; and U.S. Ser. No. 10/689,103, filed Oct. 20, 2003 and entitled “Reference Frame Attachment, the entire contents of each of which are incorporated herein by reference as are all documents incorporated by reference therein.

In a preferred embodiment, orientation of the elements on a particular location indicium varies from one location indicium to the next so that sensors according to the present invention may distinguish between various components to which the location indicia are attached in order to correlate for display and other purposes data files or images of the components. In a preferred embodiment of the present invention, some location indicia use reflective elements and some use active elements, both of which may be tracked by preferably two, sometimes more infrared sensors whose output may be processed in concert to geometrically calculate position and orientation of the item to which the location indicium is attached.

Position/orientation tracking sensors and location indicia need not be confined to the infrared spectrum. Any electromagnetic, electrostatic, light, sound, radiofrequency or other desired technique may be used. Alternatively, each item such as a surgical implement, instrumentation component, trial component, implant component or other device may contain its own “active” location indicium such as a microchip with appropriate field sensing or position/orientation sensing functionality and communications link such as spread spectrum RF link, in order to report position and orientation of the item. Such active location indicia, or hybrid active/passive location indicia such as transponders can be implanted in the body parts or in any of the surgically related devices mentioned above, or conveniently located at their surface or otherwise as desired. Location indicia may also take the form of conventional structures such as a screw driven into a bone, or any other three dimensional item attached to another item, position and orientation of such three dimensional item able to be tracked in order to track position and orientation of body parts and surgically related items. Hybrid location indicia may be partly passive, partly active such as inductive components or transponders which respond with a certain signal or data set when queried by sensors according to the present invention.

FIG. 1 illustrates an example of a conventional computer aided system 10. As shown in FIG. 1, system 10 may include, sensor 14, computer functionality 18 (which may include memory functionality 20, processing functionality 22 and input/output functionality 24), display 30, projector 32, other output device 34, foot pedal 26, imaging device 28, surgical references 16, marking device 38 and/or cutting device 40. System 10 does not require all of these items; systems 10 according to various embodiments of the present invention may have other combinations of these or other items. For example, in a preferred embodiment of the present invention, it is not necessary to use the foot pedal 26 or, if desired, for instance, display 30.

In the embodiment shown in FIG. 1, system 10 includes a computer aided surgical navigation system 12, such as the TREON™, ION™ or VECTORVISION™ systems. Computer aided surgical navigation system 12 may include a sensor 14 and computer functionality 18. Sensor 14 may be any suitable sensor, such as the ones described above or other sensors, capable of detecting the position and/or orientation of surgical references 16. In a preferred embodiment, sensor 14 emits infrared light and detects reflected infrared light to sense the position and/or orientation of surgical references 16.

Surgical reference 16 may be any device that can be secured to a structure to be referenced and detected by a sensor 14 such that the position and/or orientation of the surgical reference 16 can be detected. Suitable surgical references 16 may include, but are not limited to, location indicia secured to the bony anatomy by a pin or screw; modular location indicia secured to a platform or other structure; magnetic location indicia; quick release location indicia; adjustable location indicia; electromagnetic emitters; radio frequency emitters; LED emitters or any other surgical reference suitable for tracking by a computer assisted surgical navigation system. These and other suitable surgical references 16 are described in the documents incorporated by reference into this document.

In the embodiment shown in FIG. 1, sensor 14 may communicate information to the computer functionality 18 corresponding to the position and orientation of a surgical reference 16. Computer functionality 18, using memory functionality 20 and/or processing functionality 22 may then calculate the position and/or orientation of the structure to be referenced associated with the surgical reference 16 based on the sensed position and orientation of the surgical reference 16.

In the embodiment shown in FIG. 1, surgical references 16 are associated with structures to be referenced including an individual's body part 36 (including bony anatomy 42 and skin proximate the bony anatomy 44), marking device 38 and cutting device 40. For example, surgical reference 16 may be associated with the bony anatomy 42 and proximate skin 44 by first securely fastening surgical reference 16 to the bony anatomy 42. This may be done in any suitable and/or desirable manner, including securing the surgical reference 16 to the bony anatomy 42 in ways described above. Subsequently, imaging, such as fluoroscopy, X-ray, or other information corresponding to the bony anatomy 42, proximate skin 44 and other structure may be obtained and associated with the position and/or orientation of the surgical reference 16 secured to the bony anatomy 42. As shown in FIG. 1, such information may be obtained and associated using an imaging device 28, such as a fluoroscope associated with another surgical reference 16, or may be obtained by any other desirable and/or suitable method. Associating surgical reference 16 with the bony anatomy 42 and proximate skin 44 in this manner may allow system 10 to track and display the position and orientation of bony anatomy 42 and proximate skin 44 based on the sensed position and orientation of surgical reference 16.

Surgical references 16 may also be associated with other items, such as the cutting device 40 shown in FIG. 1, which the computer functionality 18 already has information on, such as wire-frame data. In such circumstances, a probe or other suitable device may be used to register the position and orientation of the surgical reference into the computer aided surgical navigation system allowing the position and/or orientation of the marking device 38 or cutting device 40 to be associated with the sensed position and orientation of the surgical reference 16. In some embodiments of the present invention, it is only necessary to track the position of the incision device. In some preferred embodiments, the tip of the incision device is what is tracked and compared with the suggested incision. In other embodiments, it may be preferable to track the position and orientation of the incision device. For example, it may be desirable to have the cutting device 40 enter the skin 44 at a certain angle. In such embodiments, it may be desirable to track the position and orientation of the cutting device 40 such that the entry angle of the cutting device 40 can be determined. It is also possible to superimpose images created by computer files of constructs, tools, or other items which are not actually in the surgical field; for instance, it is possible using apparatuses and methods according to aspects of the invention to overlay wire frame or other representations of cutting blocks, implants, and other components on the renderings shown on display 30 and shown or referred to using rendering apparatus, even though such components have not been introduced into the surgical field.

FIGS. 2 and 3 illustrate one particular system among the many which exist according to certain embodiments of the present invention including a rendering apparatus 220 adapted to display information on a presentation substrate, a first plurality of location indicia 230 attached to a first item used in surgery, a second plurality of location indicia 232 attached to a second item used in surgery, a sensor 250 adapted to sense the position of the first and second plurality of location indicia 230, 232, a computer functionality 260 adapted to receive information from the sensor 250 and adapted to control the movement of the rendering apparatus 220, and a monitoring apparatus 280 adapted to monitor the position of the rendering apparatus 220. According to certain aspects of some embodiments, the monitoring apparatus 280 may comprise part of the rendering apparatus 220 or may comprise part of the sensor 250. According to other embodiments, the monitoring apparatus 280 may comprise a separate apparatus. For illustration purposes in FIG. 2, the monitoring apparatus 280 is shown as a separate apparatus. While the present figure shows an embodiment with multiple items used in surgery and multiple sets of indicia, the present invention may comprise systems using only one set of location indicia or one item used in surgery. Additionally, while the computer functionality 260 and the sensor 250 are shown as separate devices, they can comprise the same device and/or comprise the same devices as the computer functionality 18 from FIG. 1 or the sensor 14 from FIG. 1.

The rendering apparatus 220 according to certain embodiments can be a laser display apparatus capable of generating or projecting a laser image directly onto one or more presentation substrates. According to other embodiments, the rendering apparatus 220 can comprise a projector, imaging device, or any other suitable rendering apparatus capable of projecting an image onto a desired substrate. The presentation substrates may comprise body parts, surgical instruments, surgical implants, display screens, or any other suitable item. In FIG. 2, the rendering apparatus 220 generates an image onto an interior surface of a patient's leg 240 and a top surface of a surgical instrument 242. The first plurality of location indicia 230, according to the depicted embodiment, comprise location indicia attached to the first item used in surgery. In FIG. 2, for purposes of illustration, the first item used in surgery is the patient's leg 240.

The first plurality of location indicia 230 can be registered with the sensor 250 and coordinated with a set of data regarding the structure of the first item used in surgery such that the computer functionality 260 can receive position information from the sensor 250 regarding the position and orientation of the first plurality of location indicia 230 and determine the position and orientation of the first item used in surgery. For example, according to the embodiment depicted in FIG. 2 for illustration purposes, the first plurality of location indicia 230 is attached to the patient's leg 240. The position of the first set of location indicia can then be correlated with, for example, an x-ray and other measurements of a tibia and fibia comprising the patient's leg 240. Once the first plurality of location indicia 230 is correlated with the x-ray and measurements associated with the patient's leg 240, the computer functionality 260 will “know” the position and orientation of the patient's leg 240 as long as the first plurality of location indicia 230 remains attached. Thus, as the patient's leg 240 is placed in dorsiflexion, extension, rotation, abduction, adduction, or anteversion, the computer functionality 260 “knows” the new position and orientation of the patient's leg 240.

The second plurality of location indicia 232, depicted in FIG. 2 are attached to a surgical instrument 242, may similarly be registered with the sensor 250 and correlated with a set of data regarding the dimensions and orientation of the surgical instrument 242. Thus, in use, the computer functionality 260 will similarly “know” the position and orientation of the surgical instrument 242 based on the position and orientation of the second set of indicia as the instrument is moved in degree of rotational or directional function. The monitoring apparatus 280 is further capable of sensing the position and/or orientation of the rendering apparatus 220. The position and/or orientation of the rendering apparatus 220, according to some embodiments, is then communicated to a computer functionality 260. The computer functionality 260 is capable of receiving information about the position and/or orientation of the rendering apparatus 220 and is further capable of controlling the position and/or orientation of the rendering apparatus 220 such that it can determine where an image projected by the rendering apparatus 220 will appear. In use, the computer functionality 260 can coordinate the position and orientation of the rendering apparatus 220 with the position and orientation of the items used in surgery so that an image projected by the rendering apparatus 220 is formed on the items used in surgery. For example, in FIG. 2, the computer functionality 260 receives information from monitoring apparatus 280 regarding the position and orientation of the rendering apparatus 220 and receives from the sensor 250 information regarding the position and orientation of the first plurality of location indicia 230 attached to a patient's leg 240.

The computer functionality 260 then determines the exact position and orientation of the anterior surface of the patient's leg 240 and adjusts the position and orientation of the rendering apparatus 220 so that an image 270 will form on the anterior surface of the patient's leg 240. Because the image is displayed onto the anterior surface of the patient's leg 240, a surgeon can perform a procedure on the patient's leg 240 and simultaneously view the image 270 displayed on the leg.

In use, the image 270 displayed by the rendering apparatus 220 may comprise data regarding the position and orientation of the patient's leg 240; including for example, an abduction angle, and an anteversion angle; a depth or angle of a planned incision; an orientation or angle of a surgical device; a plurality of vital statistics for a patient; or any other data. The rendering apparatus 220 can also render display information such as an image 274 onto the surgical instrument 242. In FIGS. 2 and 3, for illustration purposes, the image 274 displayed onto the surgical instrument 242 comprises a direction indicator representing, for example, the position and orientation of the surgical instrument 242. This information can help a surgeon achieve the desired positioning of the surgical instrument and thus avoid surgical error caused by a misaligned or malpositioned instrument.

Further capabilities of the particular system of FIGS. 2 and 3 are also shown in FIG. 3. The rendering apparatus 220 is further capable of displaying interaction indicia, such as a menu 272 onto a presentation substrate. For purposes of illustration, the presentation substrate depicted in FIG. 3 is the anterior surface of the patient's leg 240. Other suitable presentation substrates include a display screen, a surgical instrument 242, an operating table, or any other suitable surface or substrate.

The computer functionality 260 can determine from a set of data indicating the position of the menu 272, and from a set of data indicating the position of an item used in surgery, which menu choices are selected. For example, the menu 272 may contain additional indication indicia, such as, a set of prompts corresponding to a set of alternative surgical procedure plans. In order to select one of the alternative surgical plans, a surgeon may simply position the surgical instrument 242, or other device being tracked by the sensor 250, over the interaction indicia corresponding to a desired selection. As the surgical instrument 242, or other device being tracked, is positioned over the interaction indicia corresponding to the desired selection, the computer functionality 260 determines the relative position of the surgical instrument 242 with respect to the interaction indicia. The computer functionality 260 can then determine over which interaction indicia the surgeon has positioned the surgical instrument 242. The computer functionality 260 can then determine which selection the surgeon has made and can display data relating to that selection or perform any other action corresponding to the selection such as retrieving information or updating stored data. This allows a surgeon to select which data is displayed without looking up from the surgical site and without risk of contamination from contact with a data entry mechanism. Additionally, the rendering apparatus 220 may present a set of buttons for making selections, scrollbars, menu items, an image of a keyboard or number pad, or any other interaction indicia capable of input into the computer functionality 260 or other system component.

Another example of interaction indicia is depicted in FIG. 3. According to certain aspects of the embodiment depicted in FIG. 3, interaction indicia, such as a control 276, corresponding to a desired distance can be displayed. The example of the control 276 depicted in FIG. 2 comprises data relating to the desired distance and a left and a right direction indicator, which may be selected by positioning the surgical instrument 242 on or around an area on which one of the directional indicators is displayed. For example, when the surgical instrument 242, or other device whose position can be monitored by the present system, is positioned on or around the area on which the left arrow is displayed, the desired distance can be reduced by a certain amount. Alternatively, if the surgical instrument 242 or other device is positioned on or about the area on which the right directional indicator is displayed, the desired distance may be increased by a certain amount. Other interaction indicia can include, for example, scroll bars, dials, drop-down lists, alpha-numeric buttons, or any other control or interface.

While the above description contains many specifics, these specifics should not be construed as limitations on the scope of the invention, but merely as examples of the disclosed embodiments. Those skilled in the art will envision many other possible variations that are within the scope of the invention.

1. A computer assisted surgery system including sensor apparatus for sensing position and orientation of a plurality of location indicia to which surgical items and body parts are connected and computer functionality for tracking said position and orientation of said surgical items and body parts, the system further comprising:
rendering apparatus associated with the computer functionality, the rendering apparatus adapted to render display information on a presentation substrate, the presentation substrate connected to at least one location indicium adapted to be tracked by said sensor apparatus, wherein the computer functionality uses information from the sensor apparatus to track the position and orientation of the presentation substrate and cause the rendering apparatus to display the display information on the presentation substrate as the presentation substrate moves and is sensed by the sensor apparatus.

2. The computer assisted surgery system as in claim 1, wherein the presentation substrate comprises a body part.

3. The computer assisted surgery system as in claim 1, wherein the presentation substrate comprises a surgical instrument.

4. The computer assisted surgery system as in claim 1, wherein the presentation substrate comprises a display surface.

5. The computer assisted surgery system as in claim 1, wherein the rendering apparatus is further adapted to display a plurality of interaction indicia on the presentation substrate, wherein the computer functionality further uses information from the sensor apparatus to track the position and orientation of the presentation substrate and cause the rendering apparatus to display the interaction indicia on the presentation substrate as the presentation substrate moves and is sensed by the sensor apparatus; and further comprising:
a monitoring apparatus associated with the computer assisted surgery system of claim 1 adapted to monitor interaction with the interaction indicia, wherein the computer functionality further causes the monitoring apparatus to track position and location of the interaction indicia in order to monitor interaction with interaction indicia.

6. The computer assisted surgery system of claim 5, wherein the rendering apparatus comprises the monitoring apparatus.

7. The computer assisted surgery system of claim 5, wherein the monitoring apparatus comprises the sensor apparatus.

8. The computer assisted surgery system of claim 5, wherein the location indicia are fiducials.

9. The computer assisted surgery system of claim 1, wherein the rendering apparatus includes a laser projector.

10. The computer assisted surgery system of claim 5, wherein the rendering apparatus displays a graphical user interface.

11. The computer assisted surgery system of claim 10, wherein the graphical user interface comprises at least one pull down menu.

12. The computer assisted surgery system of claim 10, wherein the graphical user interface comprises at least one button.

13. The computer assisted surgery system of claim 10, wherein the graphical user interface comprises an arrangement of letters.

14. The computer assisted surgery system of claim 10, wherein the graphical user interface comprises an arrangement of numbers.

15. A computer assisted surgery system including sensor apparatus for sensing position and orientation of a plurality of location indicia to which surgical items and body parts are connected and computer functionality for tracking said position and orientation of said surgical items and body parts, the system further comprising:
rendering apparatus associated with the computer functionality, the rendering apparatus adapted to render interaction indicia on a presentation substrate during surgery, the presentation substrate connected to at least one location indicium adapted to be tracked by said sensor apparatus, wherein the computer functionality uses information from the sensor apparatus to track the position and orientation of the presentation substrate and cause the rendering apparatus to display the interaction indicia on the presentation substrate as the presentation substrate moves and is sensed by the sensor apparatus; and
a monitoring apparatus associated with the computer assisted surgery system and adapted to monitor interaction with the interaction indicia, wherein the computer functionality further causes the monitoring apparatus to track position and location of the interaction indicia in order to monitor interaction with the interaction indicia.

16. The computer assisted surgery system as in claim 15, wherein the presentation substrate comprises a body part.

17. The computer assisted surgery system as in claim 15, wherein the presentation substrate comprises a surgical instrument.

18. The computer assisted surgery system as in claim 15, wherein the presentation substrate comprises a display surface.

19. The computer assisted surgery system of claim 15, wherein the rendering apparatus comprises the monitoring apparatus.

20. The computer assisted surgery system of claim 15, wherein the monitoring apparatus comprises the sensor apparatus.

21. The computer assisted surgery system of claim 15, wherein the location indicia are fiducials.

22. The computer assisted surgery system of claim 15, wherein the rendering apparatus includes a laser projector.

23. The computer assisted surgery system of claim 15, wherein the rendering apparatus displays a graphical user interface.

24. The computer assisted surgery system of claim 23, wherein the graphical user interface comprises at least one pull down menu.

25. The computer assisted surgery system of claim 23, wherein the graphical user interface comprises at least one button.

26. The computer assisted surgery system of claim 23, wherein the graphical user interface comprises an arrangement of letters.

27. The computer assisted surgery system of claim 23, wherein the graphical user interface comprises an arrangement of numbers.

28. A computer assisted surgery system comprising
a rendering apparatus associated with the computer functionality, the rendering apparatus adapted to render display information and interaction indicia on a presentation substrate;
a first plurality of location indicia attached to the presentation substrate;
a second plurality of location indicia attached to an item used in surgery; and
a sensor apparatus adapted to sense position and orientation of the rendering apparatus, the position and orientation of the first plurality of location indicia attached to the presentation substrate; and the position and orientation of the second plurality of indicia attached to the item used in surgery, wherein the position and orientation of the rendering apparatus is coordinated with the position and orientation of the presentation substrate so that the display information and interaction indicia can be rendered on the presentation substrate, and wherein the position of the item used in surgery relative to the interaction inputs data to the computer functionality.

29. A method of performing computer assisted surgery, comprising:
providing a computer assisted surgery system including sensor apparatus for sensing position and orientation of a plurality of location indicia to which surgical items and body parts are connected and computer functionality for tracking said position and orientation of said surgical items and body parts;
providing a rendering apparatus associated with the computer functionality, the rendering apparatus adapted to render display information on a presentation substrate, the presentation substrate connected to at least one location indicium adapted to be tracked by said sensor apparatus, wherein the computer functionality uses information from the sensor apparatus to track the position and orientation of the presentation substrate and cause the rendering apparatus to display the display information on the presentation substrate as the presentation substrate moves and is sensed by the sensor apparatus;
referencing the display information from the rendering apparatus to receive data during a surgical procedure; and
completing the surgical procedure based in part on the data received from the displaying functionality.

30. A method of performing computer assisted surgery, comprising:
providing a computer assisted surgery system including sensor apparatus for sensing position and orientation of a plurality of location indicia to which surgical items and body parts are connected and computer functionality for tracking said position and orientation of said surgical items and body parts;
providing a rendering apparatus associated with the computer functionality, the rendering apparatus adapted to render interaction indicia on a presentation substrate during surgery, the presentation substrate connected to at least one location indicium adapted to be tracked by said sensor apparatus, wherein the computer functionality uses information from the sensor apparatus to track the position and orientation of the presentation substrate and cause the rendering apparatus to display the interaction indicia on the presentation substrate as the presentation substrate moves and is sensed by the sensor apparatus;
providing a monitoring apparatus associated with the computer assisted surgery system and adapted to monitor interaction with the interaction indicia, wherein the computer functionality further causes the monitoring apparatus to track position and location of the interaction indicia in order to monitor interaction with the interaction indicia;
communicating data to the computer functionality during a surgical procedure based at least on part on positioning one of the surgical items connected to a plurality of location indicia to correspond with one or more interaction indicia; and
completing the surgical procedure based at least in part on the data communicated to the computer functionality.
